(19) United States
(12) Patent Application Publication
(10) Pub. No.: US 2020/0155715 A1
(43) Pub. Date: May 21, 2020

(54) RADIOACTIVE MICROSPHERE, PREPARATION METHOD THEREOF AND RADIOACTIVE FILLER COMPOSITION

(71) Applicant: PLATINUM OPTICS TECHNOLOGY INC., Taoyuan City (TW)

(72) Inventors: Yu-Yu Tsai, Taoyuan City (TW); Fu-Yu Chang, Taoyuan City (TW); Chien-Liang Liu, Taoyuan City (TW)

(21) Appl. No.: 16/686,429

(22) Filed: Nov. 18, 2019

(30) Foreign Application Priority Data
Nov. 21, 2018 (TW) 107141463

(51) Int. Cl. A61K 51/12; A61L 27/54; A61L 27/34; A61L 27/58; A61L 27/12; A61L 27/02; A61L 27/18; A61L 27/20; A61L 27/16; C03C 3/062; C03C 4/00; C03C 11/00; C03B 19/10; C03C 23/00

(52) U.S. Cl. CPC A61K 51/1255; C03C 2204/00; A61L 27/34; A61L 27/58; A61L 27/12; A61L 27/025; A61L 27/18; A61L 27/20; A61L 27/16; C03C 3/062; C03C 4/0014; C03C 4/0042; C03C 11/002; C03B 19/102; C03C 23/0045; A61L 2430/02; A61L 2420/02; A61L 2300/622; A61L 2300/604; A61L 2300/44; A61L 27/54

(57) ABSTRACT

Provided is a radioactive microsphere including glass having a structure represented by a formula Ca₃Si₂O₇ and yttrium oxide contained in the glass. The radioactive microsphere has sphericity of from 0.71 to 1, and is radioactive after being activated by neutron irradiation. A method for preparing a radioactive microsphere and a radioactive filler composition is further provided. The present disclosure can be used to treat tumor by delivering radioactive microspheres to the target tissue, and then radioactive microspheres are activated by neutrons to generate radiation. The radioactivity of microspheres disappears over time, and the microspheres were dissolved and absorbed by the bone tissue in the end.

### BACKGROUND

### 1. Technical Field

The present disclosure relates to radioactive microspheres and fillers comprising the radioactive microspheres, and more particularly, to a radioactive filler composition for treating a bone tumor.

### 2. Description of the Prior Art

Skeletal tissues include hard bones, cartilage, ligaments and other connective tissues, with the functions of support and leverage, capability for body movement function, protection of soft tissues in the body, and storage and production of blood cells. Therefore, when the bones of a human body are damaged due to an internal disease, external injury, congenital abnormality or aging, in addition to causing inconvenience in daily life, it is more likely to cause damage to other organs.

Among the bone diseases, the more serious ones are tumors, including benign and malignant tumors. In the classification of tumors in the musculoskeletal system, only patients with Grade I tumors do not need the surgery, and the rest of patients still need the surgery in principle. In addition to the surgery, the patient with a malignant tumor requires the adjuvant therapy, including chemotherapy and radiation therapy.

A multi-mode treatment is usually used for treating a malignant tumor. Firstly, the diagnostic range is determined by precision instruments such as nuclear magnetic resonance, computed tomography and whole body bone scan. Then, the tumor samples are obtained by orthopedics, and the tumor type and grade are determined based on the pathological analysis of the tumor samples. Next, the Department of Cancer Chemotherapy and Radiation Therapy performs preoperative adjuvant therapy to control the tumor to the extent for operation, and to increase the possibility and success rate of tumor resection combined with limb preservation surgery. After completion of preoperative adjuvant therapy, the orthopedic surgery is performed by the orthopedic surgeon. In order to avoid the risk of metastasis or recurrence due to tumor remnant, the tumor is usually resected extensively. In spite of the limb reconstruction surgery, the patient is only allowed to retain the functional limb. After the resection is completed, in order to control the invisible micro metastasis, the transfer of trace residuals is often controlled by multiple postoperative adjuvant therapy. Also, the postoperative adjuvant therapy includes chemotherapy and radiation therapy.

Traditional radiation therapy uses external radiotherapy to destroy or eliminate tumors. Owing to that the radiation is greatly attenuated after being shielded by the human body, it requires to apply extremely large doses of radiation. However, these excessive doses would cause damage to normal cells of adjacent tumors at the same time.

On the other hand, bone defects caused by bone tumor resection are often filled with bone implants as a scaffold for stress and cell growth, which can effectively assist the regeneration and repair of bone tissue structure and function. At present, bone filling materials used in medical field are classified as autograft, allograft, xerograft, and synthetic graft materials. Synthetic artificial bones contain biologically active (Bioactive) materials (such as hydroxyapatite, biomedical glass, and biomedical glass ceramics) and bio-resorbable materials (such as calcium sulfate, calcium phosphate, calcium carbonate, collagen and poly-lactic acid). The bio-absorbable material, in addition to its rich source of raw materials, and no doubts about the rejection and infection of biologically derived products, can be absorbed and utilized by the original bone tissue when filling the bone tumor defect. After the healing, the strength and function of the original bone tissue would be restored.

In order to avoid the extensive resection of the tumor due to concerns about tumor remnant, to retain more available limb sites, and to reduce the inconvenience of subsequent chemotherapy and radiotherapy for tumor resection, the present disclosure utilizes absorbable artificial bone to fill the bone defect after bone tumor resection by injecting and filling, accelerating the regeneration of bone tissue after tumor resection. Meanwhile, radioactive microspheres are added to the absorbable artificial bone filling material, and owing to the characteristics of tumor vascular proliferation, the microspheres are delivered to the target residual bone tumor tissue for radiation ablation or treatment. As the residence time of the microspheres increases, the radioactive elements will gradually decay, eventually losing radioactivity and becoming harmless microspheres that remain in the body. The microspheres are used with the absorbable artificial bone filling material and eventually degrade. With being dissolved and absorbed in the bone tissue, new bones are thus formed for bone mineralization.

### SUMMARY

The present disclosure provides a radioactive microsphere comprising glass represented by the chemical formula Ca₃Si₂O₇ and yttrium oxide (Y₂O₃). The radioactive microsphere has sphericity of from 0.71 to 1, and is radioactive after being activated by neutron irradiation.

The present disclosure also provides a radioactive microsphere filler comprising a radioactive microsphere and an absorbable artificial bone filling material.

The present disclosure provides a method for preparing a radioactive microsphere, comprising: uniformly mixing glass powder represented by the chemical formula Ca₃Si₂O₇ with yttrium oxide powder, and melting at a high temperature to form glass; performing powder grinding, and then forming glass microsphere by flame spray, wherein the radioactive microsphere has sphericity of from 0.71 to 1, and is radioactive after being irradiated by neutron activation.

According to the present disclosure, a radioactive microsphere is provided, which is radioactive after being activated by neutron irradiation, and can be used to treat the tumor. Also, the present disclosure provides a radioactive microsphere filler where radioactive microspheres are added to an absorbable artificial bone filling material. By filling the absorbable artificial bone filling material into a bone defect site after tumor resection, the microspheres can be delivered to the target residual bone tumor tissue for radiation ablation or treatment. The radioactivity disappears as the microsphere residence time increases, and the harmless microspheres remain in the body, and finally degrade. The microspheres and absorbable artificial bone filling material form new bones by bone mineralization after dissolving and absorbing in the bone tissue. Consequently, the problem of performing extensive resection of the tumor due to concerns about the tumor remnant, and the inconvenience of subsequent chemotherapy and radiotherapy for surgery are significantly improved.

### BRIEF DESCRIPTION OF THE DRAWINGS

FIG. 1 is a scanning electron microscope (SEM) photograph of radioactive microspheres; and

FIG. 2 is a scanning electron microscope (SEM) photograph of the radioactive microspheres.

### DETAILED DESCRIPTION OF THE EMBODIMENTS

The following embodiments are intended to illustrate the disclosure of the present disclosure. After reading the disclosure of the specification, those skilled in the art can easily understand the advantages and functions thereof.

It shall be understood that the structure, the proportions, the dimensions, and the like of the present disclosure are intended to enhance the understanding and perusal by those skilled in the art, and are not intended to limit the present disclosure with the specific conditions. Accordingly, they do not have technical significance. Any changes in the structure, changes in the proportional relationship, or adjustments in the dimensions are intended to be included in the scope of the present disclosure, without affecting the effects and the achievable objectives of the present specification. Without substantially altering the technical contents, changes or adjustments in relative relationships are considered as fallen within the implementable scope of the present disclosure.

The present disclosure provides radioactive microspheres including glass represented by the chemical formula Ca₃Si₂O₇ and yttrium oxide contained in the glass, and the sphericity of the radioactive microspheres is from 0.71 to 1.

Ca₃Si₂O₇ is mainly formed by mixing CaO with SiO₂ and melting at a high temperature, and a molar ratio of CaO to SiO₂ is 4:6, and the temperature is at least 1400 degrees.

CaSiO₃ (wollastonite) is a typical calcium-based biomaterial capable of forming a calcium phosphate layer and an yttrium-rich layer in SBF (simulated body fluid), produces hydroxyapatite (HA) with osteo-conductive and osteo-inductive biological activity, and has better biological activity and degradability than HA. The types of CaSiO₃ include α-wollastonite (Ca₂SiO₄), α′-wollastonite (Ca₂SiO₄), β-wollastonite (pseudo wollastonite; Ca₃Si₃O₉), hatrurite (Ca₃SiO₅), and rankinite (Ca₃Si₂O₇), wherein Ca₃Si₂O₇ exhibits glass phase.

The sphericity (Ψ) used in the present disclosure is calculated by Wadell sphericity, and the formula thereof is:

$$Ψ = \frac{{A}_{s}}{{A}_{p}} = \frac{{{π}^{1 / 3} ( 6 {V}_{p} )}^{2 / 3}}{{A}_{p}} = \frac{\sqrt[3]{36 π {V}_{p}^{2}}}{{A}_{p}} \qquad ( Formula I )$$

Wherein Aₛ is the equivalent spherical surface area (an equivalent sphere, that is, the sphere whose volume is the same as the object to be tested), Aₚ is the surface area of the object to be tested, and Vₚ is the volume of the object to be tested.

In an embodiment, the glass sphere further includes an imaging nuclide oxide. Before neutron activation, the imaging nuclide oxide is at least selected from the group consisting of phosphorus, calcium, sodium, rhenium, scandium, lanthanum, cerium, praseodymium, neodymium, promethium, samarium, europium, gadolinium, terbium, dysprosium, holmium, erbium, thulium, ytterbium, lutetium, actinium-225, antimony-127, arsenic-74, barium-140, bismuth-210, californium-246, calcium-46, calcium-47, carbon-11, carbon-14, cesium-131, cesium-137, chromium-51, cobalt-57, cobalt-58, cobalt-60, dysprosium-165, erbium-169, fluorine-18, gallium-67, gallium-68, gold-198, holmium-166, hydrogen-3, indium-111, indium-113m, iodine-123, iodine-125, iodine-131, iridium-192, iron-59, iron-82, krypton-81m, lanthanum-140, lutetium-177, molybdenum-99, nitrogen-13, oxygen-15, palladium-103, phosphorus-32, radon-222, radium-224, rhenium-186, rhenium-188, rhodium-82, samarium-153, selenium-75, sodium-22, sodium-24, strontium-89, technetium-99m, thallium-201, xenon-127, xenon-133 and yttrium-90. The imaging nuclide oxide is then activated by neutron activation and decays into an element represented in the brackets: phosphorus (³²P->³²S), calcium (⁴⁷Ca->⁴⁷Sc; ⁴⁹Ca->⁴⁹Sc), sodium (²²Na->²²Ne), rhenium (¹⁸⁸Re->¹⁸⁸Os), scandium (⁴⁴Sc->⁴⁴Ca; ⁴⁸Sc->⁴⁸Ti; ⁴⁶Sc->⁴⁶Ti; ⁴⁷Sc->⁴⁷Y), lanthanum (¹⁴⁰La->¹⁴⁰Ce; ¹⁴²La->¹⁴²Ce), cerium, praseodymium, neodymium, promethium, samarium, europium, gadolinium, terbium, dysprosium, holmium, erbium, thulium, ytterbium, lutetium, actinium-225(²²⁵Ac->²²¹Fr, ²¹¹Bi,¹⁴C), antimony-127(¹²⁷Sb->¹²⁷Te), arsenic-74(⁷⁴As->⁷⁴Ge), barium-140(¹⁴⁰Ba->¹⁴⁰La), bismuth-210(²¹⁰Bi->²¹⁰Po), californium-246(²⁴⁶Cf->²⁴⁶Cm), calcium-46(⁴⁶Ca->⁴⁶Sc), calcium-47(⁴⁷Ca->⁴⁷Sc), carbon-11(¹¹C->¹¹B), carbon-14(¹⁴C->¹⁴N), cesium-131(¹³¹Cs->¹³¹Xe;¹³¹Cs->¹³¹Ba), cesium-137(¹³⁷Cs->¹³⁷Ba), chromium-51(⁵¹Cr->⁵¹V), cobalt-57(⁵⁷Co->⁵⁷Fe), cobalt-58(⁵⁸Co->⁵⁸Fe), cobalt-60(⁶⁰Co->⁶⁰Ni), dysprosium-165(¹⁶⁵Dy->¹⁶⁵Ho), erbium-169(¹⁶⁹Er->¹⁶⁹Tm), fluorine-18(¹⁸F->¹⁸O), gallium-67(⁶⁷Ga->⁶⁷Zn), gallium-68(⁶⁸Ga->⁶⁸Zn), gold-198(¹⁹⁸Au->¹⁹⁸Hg), holmium-166(¹⁶⁶Ho->¹⁶⁶Er), hydrogen-3(³H->³He), indium-111(¹¹¹In->¹¹¹Cd), indium-113m (^{113m}In->¹¹³Sn), iodine-123(₁₂₃I->¹²³Te), iodine-125(₁₂₅I->₁₂₅Te), iodine-131(¹³¹I->¹³¹Xe), iridium-192 (¹⁹²Ir->¹⁹²Os,¹⁹²Pt), iron-59(⁵⁹Fe->⁵⁹Co), krypton-81m (^{81m}Kr->⁸¹Br), lanthanum-140(¹⁴⁰La->¹⁴⁰Ce), lutetium-177(¹⁷⁷Lu->¹⁷⁷Hf), molybdenum-99(⁹⁹Mo->⁹⁹Tc,⁹⁹Ru), nitrogen-13(¹³N->¹³C), oxygen-15(¹⁵O->¹⁵N), palladium-103(¹⁰³Pd->¹⁰³Rh), Phosphorus-32(³²P->³²S), radon-222(²²²Rn->²¹⁸Po), radium-224(²²⁴Ra->²²⁰Rn,²¹⁰Pb,¹⁴C), rhenium-186(¹⁸⁶Re->¹⁸⁶Os,¹⁸⁶W), rhenium-188(¹⁸⁸Re->¹⁸⁸Os), samarium-153(¹⁵³Sm->¹⁵³Eu), selenium-75 (⁷⁵Se->⁷⁵As), sodium-22(²²Na->²²Ne), sodium-24(²⁴Na->²⁴Mg), strontium-89(⁸⁹Sr->⁸⁹Y), technetium-99m (⁹⁹Tc->⁹⁹Ru), thallium-201(²⁰¹Tl->²⁰¹Hg), xenon-127(¹²⁷Xe->¹²⁷Cs), xenon-133(¹³³Xe->¹³³Cs) and yttrium-90(⁹⁰Y->⁹⁰Zr).

In an embodiment, the imaging nuclide oxide is in an amount of from 0 to 10% by weight, more preferably, from 3 to 8% by weight, in the radioactive microspheres.

The imaging nuclide oxide used in the present disclosure emits y-rays after being activated by neutron irradiation, and can be detected and distributed in vivo through special photographic equipment such as y camera or positron tomography. The integration of these photographic devices with computers can display images and can be calculated and analyzed for more information. Since most diseases have physiological, biochemical and metabolic changes in the early stages of the disease, and then structural changes, X-ray inspection and computerized tomography are those commonly used to detect the body structure changes. In addition, the nuclear angiography can detect abnormalities before the onset of the disease and the presence of symptoms in other examination methods, because it can show the physiological changes of the organ tissues. This ability to diagnose early often allows the disease to be treated before the disease progresses rapidly.

In an embodiment, the particle size of the radioactive microspheres is preferably from 20 to 100 μm.

In an embodiment, the molar ratio of glass to yttrium oxide in the radioactive microspheres is preferably from 80:20 to 70:30, within which Ca₃Si₂O₇ maintains a good glass phase and has a sufficient radiation dose.

In an embodiment, the radioactive microspheres further include a coating layer formed on a surface of the glass, the coating layer comprising one of an organic material and an inorganic material, or a combination thereof.

In an embodiment, the organic material includes an acid group, a hydroxyl group, an amine group, or a carboxyl group.

In an embodiment, the organic material includes a biodegradable material.

In an embodiment, the inorganic material includes a phosphate compound, a sulfate compound, a chloride salt compound, a nitrate compound, or a borate compound.

In an embodiment, the coating layer is poly-vinyl-pyrrolidone, poly-vinyl-alcohol, carboxymethyl cellulose, polyethylene glycol (PEG6000), methylcellulose, hydroxyl-propyl methyl cellulose, hydroxyl-propyl cellulose, gum arabic, poly-L-lactic acid/poly(lactic-co-glycolic acid) (PLLA/PLGA) or Ca₃(PO₄)₂.

When the radioactive microsphere of the present disclosure remains radioactive in time and remains in the body, it does not affect the degradation of the absorbable artificial bone filling material. Also, the radioactive microsphere can become a stable structure with the absorbable artificial bone filling material before the new bone is formed, and provides an environment for bone growth. It is suitable for use as an additive to artificial bone filling materials.

The present disclosure also provides a radioactive filling composition, comprising the radioactive microsphere and an absorbable artificial bone filling material, wherein the absorbable artificial bone filling material is at least one selected from the group consisting of calcium sulfate, calcium phosphate, calcium carbonate and poly-lactic acid.

The calcium sulfate salt is one or more selected from the group consisting of calcium sulfate anhydrate, calcium sulfate hemihydrate, calcium sulfate di-hydrate, or a mixture, composition or adduct of anhydrous calcium sulfate/calcium sulfate hemihydrate, anhydrous calcium sulfate/calcium sulfate di-hydrate, calcium sulfate hemihydrate/calcium sulfate di-hydrate, or anhydrous calcium sulfate/calcium sulfate hemihydrate/calcium sulfate di-hydrate.

The calcium phosphate salt is one or more selected from the group consisting of calcium phosphate, di-calcium phosphate (DCP), tri-calcium phosphate (TCP), calcium hydrogen phosphate, tetra-calcium phosphate (TTCP), hydroxyapatite (HA), strontium hydroxyapatite, magnesium hydroxyapatite, and silver hydroxyapatite, or a mixture, composition or adduct of DCP/TCP, DCP/TTCP, DCP/HA, TCP/TTCP, TCP/HA, TTCP/HA, DCP/TCP/TTCP, DCP/TCP/HA, DCP/TTCP/HA, TCP/TTCP/HA, and DCP/TCP/TTCP/HA.

The radioactive microsphere and the absorbable artificial bone filling material are mixed with an additive and a liquid, and can be implanted and solidified after being implanted into the bone defect site after the tumor resection, providing a scaffold for stress and cell growth and locally killing the residual cancer. Distribution may be observed using imaging nuclide.

The additive is one or more selected from polyethylene glycol, sodium alginate, polyvinyl alcohol, cellulose, chitosan, hyaluronic acid, sodium stearate, magnesium stearate, gelatin, preferably one or more of polyethylene glycol, sodium alginate, hyaluronic acid, chitosan, and cellulose.

The liquid is, for example, pure water, physiological saline, phosphate solution, graphene oxide solution, chitosan solution, sodium alginate solution, sodium citrate solution, sodium hyaluronate solution, polyvinyl alcohol solution, polyethylene glycol solution, cellulose solution, silver nitrate solution, cellulose solution, artificial body fluid or human blood. Preferably, it is 0.05 to 3% by weight of a sodium hyaluronate solution, 0.05 to 3% by weight of a chitosan solution, 0.05 to 3% by weight of a sodium alginate solution, water or blood.

The present disclosure also provides a method for preparing a radioactive microsphere, including steps of melting a mixture comprising glass powder represented by the chemical formula Ca₃Si₂O₇ and yttrium oxide powder to form glass; cooling the glass; grinding the glass to obtain glass fine grains; and flame-spraying the glass fine grains to form a radioactive microsphere, wherein the radioactive microsphere has sphericity of from 0.71 to 1.

In an embodiment, the glass fine grains are flame-sprayed to form a radioactive microsphere, and then collected in a cooling collection zone.

The cooling collection zone may be a solid or liquid interface, the solid may be ice or dry ice, and the liquid may be a liquid that can excite for a nuclide component (an organic/inorganic acid) or water.

In an embodiment, the method of preparing the radioactive microspheres further comprises adding an imaging nuclide oxide powder to the mixture prior to melting the mixture.

In an embodiment, the method for preparing the radioactive microspheres further comprises forming a coating layer on the surface of the radioactive microspheres.

Specifically, the mixed powder is pre-ball milled and uniformly mixed, and then melted to form glass; after grinding the glass, the glass fine grains are obtained. Said glass fine grains are heated and sprayed by high-speed gas flame. Specifically, the glass fine grains are heated by the high-temperature combustion flame, with the high-speed combustion gas spraying away from the flame core, to cause the surface melting, and under the influence of surface tension interaction, high-temperature molten droplets are formed. During the rotating flight, the high-temperature molten droplets gradually form a spherical shape due to the influence of air temperature gradient, gravity and droplet rotation, and eventually contact the cooling collection zone when the distance from the flame center is increased. The temperature gradient of the cooling collection zone is sharply reduced to form a radioactive microsphere. On the other hand, under different processing conditions of different flight distances and different nature flames, the resulting shape would form a solid sphere, a hollow sphere or a mesoporous sphere as the distance of the radioactive microspheres from the flame center is different. Moreover, the composition of the flame depends on the mixing ratio of the combustion gas and the oxygen. In particular, when the mixing ratio of oxygen and acetylene in the oxidizing flame (Nm³/hr) is greater than 1.2, it is an oxygen-excess flame, which is with oxidizing property. When the mixing ratio of oxygen and acetylene in the neutral flame is 1.1 to 1.2, oxygen and acetylene are fully burned, and there is no problem of excess oxygen and acetylene; and the inner flame has a certain reducing property, so that the CO₂ and CO generated during combustion have a protective effect. When in the carbonized flame the mixing ratio of oxygen to acetylene is less than 1.1, the acetylene is excessive and has strong reducing property; and the flame has free carbon and excessive hydrogen.

The present disclosure illustrates the details by way of examples of the embodiments. However, the interpretation of the present disclosure should not be limited to the description of the following examples.

### Example (1)

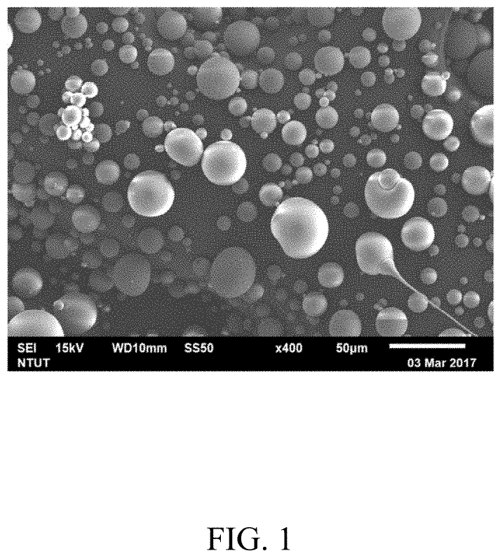

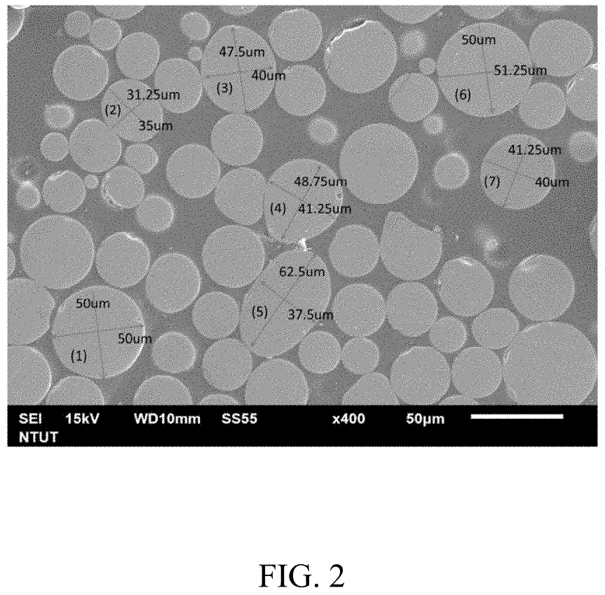

The glass powder represented by the chemical formula Ca₃Si₂O₇ and the yttrium oxide powder were uniformly ball-milled at a molar ratio of 80:20, and melted to form glass; after powder grinding, flame spraying was performed by a high-speed gas mixed with acetylene and oxygen (gas ratio of 1.1 to 1.2) for heating and spraying. The radioactive microspheres were formed at a flame temperature range of from 1200° C. to 2000° C., with a spray distance of 50 cm, and a flight time of 15 seconds. The radioactive microspheres were as shown in FIG. 1. The radioactive microspheres were subject to Wadell sphericity analysis (as shown in FIG. 2 and Table 1), and the spherical microspheres had sphericity between 0.7276 and 1.

The radioactive microspheres were taken by 10 mg for neutron activation irradiation; and after neutron activation element analysis, Ca signal was observed, as shown in Table 2.

### Example (2)

The glass powder represented by the chemical formula Ca₃Si₂O₇ and the yttrium oxide powder were uniformly ball-milled at a molar ratio of 80:20, and then imaging nuclide oxide powders such as 5% by weight of ReO, 5% by weight of CuO, and 5% by weight of TeO were separately added. After being melted to form glass, powder grinding was performed, and then flame spraying was performed by a high-speed gas (gas ratio of 1.1 to 1.2) mixed with acetylene and oxygen. The flame was sprayed at a flame temperature range of from 1200° C. to 2000° C., with a spraying distance of 50 cm and a flight time of 15 seconds, to form radioactive microspheres. The radioactive microspheres comprising ReO, CuO or TeO were taken by 10 mg and irradiated for neutron activation. After neutron activation element analysis, the signals of Re, Cu and Te (I-131) were observed, as shown in Table 3.

### Example (3)

The glass powder represented by the chemical formula Ca₃Si₂O₇ and the yttrium oxide powder were uniformly ball-milled at a molar ratio of 80:20, and then imaging nuclide oxide powders such as 5% by weight of ReO, 5% by weight of CuO, and 5% by weight of TeO were separately added. After being melted to form glass, powder grinding was performed, and then flame spraying was performed by a high-speed gas (gas ratio of 1.1 to 1.2) mixed with acetylene and oxygen. The flame was sprayed at a flame temperature range of from 1200° C. to 2000° C., with a spraying distance of 50 cm and flight time of 15 seconds, to form radioactive microspheres. The radioactive microspheres were mixed with 10 g of calcium sulfate hemihydrate and 0.5 g of magnesium stearate at room temperature, and then were stirred with 3 g of pure water as a mixed liquid. The results were all moldable and curable.

### Example (4)

The glass powder represented by the chemical formula Ca₃Si₂O₇ and the yttrium oxide powder were uniformly ball-milled at a molar ratio of 80:20, and then imaging nuclide oxide powders such as 5% by weight of ReO, 5% by weight of CuO, and 5% by weight of TeO were separately added. After being melted to form glass, powder grinding was performed, and then flame spraying was performed by a high-speed gas (gas ratio of 1.1 to 1.2) mixed with acetylene and oxygen, and the flame was sprayed at a flame temperature range of from 1200° C. to 2000° C., with a spraying distance of 50 cm and a flight time of 15 seconds, to form radioactive microspheres. The radioactive microspheres were mixed with 10 g of mono-calcium phosphate glass and 0.5 g of magnesium stearate additive at room temperature, and then were stirred with 3 g of PBS artificial body fluid as a mixed liquid. The results were all moldable and curable.

### Example (5)

The glass powder represented by the chemical formula Ca₃Si₂O₇ and the yttrium oxide powder were uniformly ball-milled at a molar ratio of 80:20, and then imaging nuclide oxide powders such as 5% by weight of ReO, 5% by weight of CuO, and 5% by weight of TeO were separately added. After being melted to form glass, powder grinding was performed, and then flame spraying was performed by a high-speed gas (gas ratio of 1.1 to 1.2) mixed with acetylene and oxygen, and the flame was sprayed at a flame temperature range of from 1200° C. to 2000° C., with a spraying distance of 50 cm and a flight time of 15 seconds, to form radioactive microspheres. The radioactive microspheres were respectively mixed with 10 g of a mixture mono-calcium phosphate glass and calcium sulfate hemihydrate at room temperature, where the two powders were blended at a ratio of 1:4, 1:1 and 4:1, and then the mixture was stirred with 3 g of PBS artificial body fluid. The results were all moldable and curable.

### Example (6)

The glass powder represented by the chemical formula Ca₃Si₂O₇ and the yttrium oxide powder were uniformly ball-milled at a molar ratio of 80:20, and then imaging nuclide oxide powders such as 5% by weight of ReO, 5% by weight of CuO, and 5% by weight of TeO were separately added. After being melted to form glass, powder grinding was performed, and then flame spraying was performed by a high-speed gas (gas ratio of 1.1 to 1.2) mixed with acetylene and oxygen, and the flame was sprayed at a flame temperature range of from 1200° C. to 2000° C., with a spraying distance of 50 cm and a flight time of 15 seconds, to form radioactive microspheres. The radioactive microspheres were coated with an organic or inorganic material layer (as shown in Table 4) on the outer surface of the glass microspheres by a spray granulation method at room temperature. The results show that the radioactive microspheres can be over molded.

TABLE 1
Sphericity analysis

| | | | Aₛ being | | |
|---|---|---|---|---|---|
| | Elliptical | Elliptical | equivalent | Aₚ being | |
| | long | short | spherical | surface area | |
| | axis | axis | surface | of the object | Sphericity |
| | (μm) | (μm) | area | to be tested | (Ψ) |
| (1) | 50 | 50 | 7853.98 | 7853.30 | 1.000 |
| (2) | 35 | 31.25 | 3067.9615 | 3256.964 | 0.942 |
| (3) | 47.5 | 40 | 5026.548 | 5517.946 | 0.911 |
| (4) | 48.75 | 41.25 | 5345.616 | 5851.2905 | 0.9135 |
| (5) | 62.5 | 37.5 | 4417.86467 | 6071.2508 | 0.7276 |
| (6) | 51.25 | 50 | 7853.98 | 7952.0769 | 0.9876 |
| (7) | 41.25 | 40 | 5026.548 | 5162.959 | 0.9735 |

TABLE 2
Neutron irradiation activation element analysis

| | | | | γ-ray | | | |
|---|---|---|---|---|---|---|---|
| | | | | energy | | | |
| | | | | spectrum | | | |
| | | | | main | | | |
| | | Test | | peak | | | Specific |
| | | product | Radio- | energy | AUC | | activity |
| Compound | | weight | isotope | (keV) | (cnts) | Bq | (uCi/mg) |
| 1 | Y, Ca | 10 mg | Y-90m | 202 keV | 393 | 74.1 | 0.00022 |

TABLE 3
Neutron irradiation activation element analysis

| | | | | γ-ray | | | |
|---|---|---|---|---|---|---|---|
| | | | | energy | | | |
| | | | | spectrum | | | |
| | | | | main | | | Specific |
| | | Test | | peak | | | activity |
| | | product | Radio- | energy | AUC | | (uCi/ |
| Compound | | weight | isotope | (keV) | (cnts) | Bq | mg) |
| 1 | Y, Cu | 10 mg | Y-90m | 202 keV | 410 | 66.9 | 0.00018 |
| 2 | Y, Cu | 10 mg | Y-90m | 202 keV | 393 | 64.1 | 0.00017 |
| 3 | Y, Te | 10 mg | I-131 | 364.5 keV | 20 | 3.9 | 0.00001 |
| 4 | Y, Te | 10 mg | Y-90m | 202 keV | 288 | 47.0 | 0.00013 |
| 5 | Y, Re | 10 mg | Re-188 | 155.1 keV | 1524 | 1612.7 | 0.00435 |

TABLE 4
Microsphere coated with organic and inorganic materials

| | | | Outlet | Over- |
|---|---|---|---|---|
| Test coatingmaterial | wt % | Flow rate | temperature | molding |
| Poly-vinyl-pyrrolidone | 0.1-10 | 357 L/h | 180-245° C. | Yes |
| (PVP) | | | | |
| Poly-vinyl alcohol (PVA) | 0.1-10 | 357 L/h | 180-245° C. | Yes |
| Carboxymethylcellulose | 0.1-10 | 357 L/h | 180-245° C. | Yes |
| (CMC) | | | | |
| Poly-ethyleneglycol | 0.1-10 | 357 L/h | 180-245° C. | Yes |
| (PEG 6000) | | | | |
| Methylcellulose(MC) | 0.1-10 | 357 L/h | 180-245° C. | Yes |
| Hydroxy-propyl methyl | 0.1-10 | 357 L/h | 180-245° C. | Yes |
| cellulose(HPMC) | | | | |
| Hydroxy-propyl cellulose | 0.1-10 | 357 L/h | 180-245° C. | Yes |
| (HPC) | | | | |
| Gum arabic | 0.1-10 | 357 L/h | 180-245° C. | Yes |
| Poly L-lactic acid/poly- | 0.1-10 | 357 L/h | 180-245° C. | Yes |
| lactic acid-glycolic | | | | |
| acid copolymer (PLLA/ | | | | |
| PLGA) | | | | |
| Ca₃(PO₄)₂ | 0.1-10 | 357 L/h | 180-245° C. | Yes |

What is claimed is:

1. A radioactive microsphere, comprising glass represented by a chemical formula of Ca₃Si₂O₇ and yttrium oxide contained in the glass, and having sphericity from 0.71 to 1, wherein the radioactive microsphere is radioactive after being activated by neutron irradiation.

2. The radioactive microsphere of claim 1, further comprising an imaging nuclide oxide.

3. The radioactive microsphere of claim 2, wherein the imaging nuclide oxide has an imaging nuclide that is at least one selected from the group consisting of phosphorus, calcium, sodium, rhenium, scandium, lanthanum, cerium, praseodymium, neodymium, promethium, samarium, europium, gadolinium, terbium, dysprosium, holmium, erbium, thulium, ytterbium, lutetium, actinium-225, antimony-127, arsenic-74, barium-140, bismuth-210, californium-246, calcium-46, calcium-47, carbon-11, carbon-14, cesium-131, cesium-137, chromium-51, cobalt-57, cobalt-58, cobalt-60, dysprosium-165, erbium-169, fluorine-18, gallium-67, gallium-68, gold-198, holmium-166, hydrogen-3, indium-111, indium-113m, iodine-123, iodine-125, iodine-131, iridium-192, iron-59, iron-82, krypton-81m, lanthanum-140, lutetium-177, molybdenum-99, nitrogen-13, oxygen-15, palladium-103, phosphorus-32, radon-222, radium-224, rhenium-186, rhenium-188, rhodium-82, samarium-153, selenium-75, sodium-22, sodium-24, strontium-89, technetium-99m, thallium-201, xenon-127, xenon-133 and yttrium-90.

4. The radioactive microsphere of claim 1, wherein the radioactive microsphere has a particle diameter of 20 μm to 100 μm.

5. The radioactive microsphere of claim 1, wherein a molar ratio of the glass to the yttrium oxide is from 80:20 to 70:30.

6. The radioactive microsphere of claim 1, further comprising a coating layer formed on a surface of the glass.

7. The radioactive microsphere of claim 6, wherein the coating layer comprises one of an organic material, an inorganic material, or a combination thereof.

8. The radioactive microsphere of claim 7, wherein the organic material comprises a biodegradable material and/or a residue comprising an acid group, a hydroxyl group, an amine group or a carboxyl group, and the inorganic material comprises a phosphate compound, a sulfate compound, a chloride salt compound, a nitrate compound or a borate compound.

9. The radioactive microsphere of claim 7, wherein the coating layer is poly-vinyl-pyrrolidone, poly-vinyl-alcohol, carboxymethyl cellulose, poly-ethylene glycol (PEG6000), methylcellulose, hydroxyl-propyl methyl cellulose, hydroxyl-propyl cellulose, gum arabic, poly-L-lactic acid/poly(lactic-co-glycolic acid) (PLLA/PLGA) or Ca₃(PO₄)₂.

10. A radioactive filler composition comprising the radioactive microsphere of claim 1 and an absorbable artificial bone filling material.

11. The radioactive filler composition of claim 10, wherein the absorbable artificial bone filling material is at least one selected from the group consisting of calcium sulfate, calcium phosphate, calcium carbonate and poly-lactic acid.

12. A method of preparing a radioactive microsphere, comprising:
melting a mixture comprising glass powder represented by a chemical formula Ca₃Si₂O₇ and yttrium oxide powder to form glass;
cooling the glass;
grinding the glass to obtain glass fine grains; and
flame spraying the glass fine grains to form a radioactive microsphere having sphericity of from 0.71 to 1, wherein the radioactive microsphere is radioactive after being activated by neutron irradiation.

13. The method of claim 12, further comprising adding imaging nuclide oxide powder to the mixture prior to melting the mixture.

14. The method of claim 13, wherein the imaging nuclide oxide has an imaging nuclide that is at least one selected from the group consisting of phosphorus, calcium, sodium, rhenium, scandium, lanthanum, cerium, praseodymium, neodymium, promethium, samarium, europium, gadolinium, terbium, dysprosium, holmium, erbium, thulium, ytterbium, lutetium, actinium-225, antimony-127, arsenic-74, barium-140, bismuth-210, californium-246, calcium-46, calcium-47, carbon-11, carbon-14, cesium-131, cesium-137, chromium-51, cobalt-57, cobalt-58, cobalt-60, dysprosium-165, erbium-169, fluorine-18, gallium-67, gallium-68, gold-198, holmium-166, hydrogen-3, indium-111, indium-113m, iodine-123, iodine-125, iodine-131, iridium-192, iron-59, iron-82, krypton-81m, lanthanum-140, lutetium-177, molybdenum-99, nitrogen-13, oxygen-15, palladium-103, phosphorus-32, radon-222, radium-224, rhenium-186, rhenium-188, rhodium-82, samarium-153, selenium-75, sodium-22, sodium-24, strontium-89, technetium-99m, thallium-201, xenon-127, xenon-133 and yttrium-90.

15. The method of claim 12, wherein the radioactive microsphere has a particle diameter of 20 μm to 100 μm.

16. The method of claim 12, wherein a molar ratio of the glass powder to the yttrium oxide represented by the chemical formula Ca₃Si₂O₇ is from 80:20 to 70:30.

17. The method of claim 12, further comprising forming a coating layer on a surface of the radioactive microsphere.

18. The method of claim 17, wherein the coating layer comprises one of an organic material, an inorganic material, or a combination thereof.

19. The method of claim 18, wherein the organic material comprises a bio-degradable material and/or a residue comprising an acid group, a hydroxyl group, an amine group or a carboxyl group, and the inorganic material comprises a phosphate compound, a sulfate compound, a chloride salt compound, a nitrate compound or a borate compound.

20. The method of claim 18, wherein the coating layer is poly-vinyl-pyrrolidone, poly-vinyl-alcohol, carboxymethyl cellulose, poly-ethylene glycol (PEG6000), methylcellulose, hydroxyl-propyl methyl cellulose, hydroxyl-propyl cellulose, gum arabic, poly-L-lactic acid/poly(lactic-co-glycolic acid) (PLLA/PLGA) or Ca₃(PO₄)₂.
